(19) United States
(12) Patent Application Publication
(10) Pub. No.: US 2010/0042040 A1
(43) Pub. Date: Feb. 18, 2010

(54) SYSTEM, DEVICE AND METHOD FOR THE ANTIBACTERIAL AND FUNGICIDE TREATMENT OF A HUMAN OR ANIMAL BODY

(76) Inventor: Jochen Arentz, Hamburg (DE)

Correspondence Address: ROBERTS & ROBERTS, LLP;ATTORNEYS AT LAW, P.O. BOX 484, PRINCETON, NJ, 08542-0484, US

(21) Appl. No.: 12/445,521

(22) PCT Filed: Jul. 4, 2007
(86) PCT No.: PCT/EP07/56765

§ 371 Date: Apr. 14, 2009

(30) Foreign Application Priority Data
Oct. 16, 2006 (DE) 202006016025.6

(51) Int. Cl. A61N 5/06; A61M 37/00

(52) U.S. Cl. 604/20; 607/88

(57) ABSTRACT

The invention relates to a system, a device, and a method for the antibacterial treatment of a patient, using a combination of light and dye. The invention is characterised in that the maximum intensity of the light is at a wavelength which is different to the wavelength of a maximum absorption of the dye. Such a device can be used to obtain especially good bactericide and/or fungicide results.

### FIELD OF THE INVENTION

The invention relates to a system for the antibacterial treatment of a human or animal body, in particular for treatment in the mouth and/or of wounds, containing:

a dye, in particular a dye with a phenothiazine molecular structure, in which the dye has an at least local absorption maximum and a first wavelength and at least upon application the dye is in the form of a dye solution, and

a light unit, for generating and, in particular in a directional way, outputting light onto the regions to be treated.

The invention further relates to a device as generically defined by the preamble to claim 6 and to a method as generically defined by the preamble to claims 12 and 13.

### PRIOR ART

In the field of medicine, it is already long been known to use methylene blue as a dye for staining biological tissue. In dilute concentrations, in any case, this dye is essentially nontoxic, so that it can be used largely without risk. In addition, numerous other dyes are used, such as toluidine blue-O.

In the recent past, methods have been described in which, by using laser light adapted to absorption maximums of the dye employed, in conjunction with such a dye, a bactericidal system is used. Such methods are known by the term photodynamic therapy (PDT). As dyes that can possibly be used, methylene blue and toluidine blue have been proposed as examples. One example of such a system or apparatus is described in German Patent Disclosure DE 10349710 A_. This arrangement is intended in particular to be used for germ-reducing treatment of a patient in the mouth, especially in the region of the gums. In this reference, it is described that as the laser, a semiconductor diode laser is preferably used, and methylene blue is preferably used as the dye. According to this reference, the dye is adapted in its emitted wavelength to one of the absorption maximums of the dye methylene blue and accordingly is emitted at a wavelength of 660 nanometers. The rated power of the laser as described there is 100 milliwatts. The solution containing methylene blue as the dye contains the dye in a concentration of approximately 0.01 weight %.

Although the method and apparatus described in this reference fundamentally provide good service in germ reduction or germ fighting, nevertheless, the efficiency of this apparatus can be further improved. Especially, the deficiency of this method resides in the only very slight penetration depth of the light, which is due to the strong absorption of the dye. Only with a very low concentration of dye is anything but a purely superficial effect at all possible.

Such a system has also been disclosed, with the variation of some parameters, in detail by You Chan and Chen-Hsiung Lai, in “Bactericidal effects of different laser wavelength on periodontopathic germs in photodynamic therapy”, Med. Sci. 2003, Nr. 18, pp. 51-55.

In this study, the bactericidal effect of methylene blue in a concentration of 0.01 weight % was studied in combination with laser light at the three different wavelengths of 632.8 nm, 665 nm, and 830 nm. The researchers discovered that in the case of the choice of 830 nm, only a very slight bactericidal effect occurs.

In particular, they were able neither to disclose a pronounced improvement in the PDT nor to indicate a direction in which such an improvement might be looked for. However, the article makes it clear that the choice of a comparatively long wavelength does not promise success.

### SUMMARY OF THE INVENTION

It is accordingly the object of the present invention to improve the bactericidal effect of the PDT method.

This object is attained surprisingly by a system as defined by claim 1 and/or 2, a device as defined by claim 6, and a method as defined by claim 12 and/or 13. Advantageous refinements are disclosed in dependent claims 3 through 5 and 7 through 11.

A substantial aspect of the invention resides in the special choice of the wavelength with regard to the selected dye. It is already known to choose the wavelength of the light in the range of one absorption maximum, or of the absorption maximum, of the dye. For instance, the absorption maximum of methylene blue in a 96% ethanol aqueous solution is 655 nm. The wavelength described in DE 103 49 710 A1 amounts to 660 nm, and the wavelengths that were promising of success in the study by You Chan and Chen-Hsiung Lai are 632.8 nm and 665 nm. These wavelengths are quite close to the absorption maximum of the dye used.

The inventor first analyzed the absorption behavior of the dyes (see FIG. 1 and the explanation thereof hereinafter). Based on this finding, it was possible to understand the effect of the method and to attain the stated object.

In the previously known methods, the absorption of the incident light, despite the comparatively low concentration selected, at 0.001 to 0.01 weight %, is very high in the uppermost layers of the dye solution. After only a very short distance, the entire light output is already absorbed. Thus only a slight proportion of the introduced dye can interact with the light. The very great majority of the dye, usually on its own, develops the effect that the dye has even without incident light. The bactericidal effect of the dye is negligible, because of the low concentration.

By the choice of higher dye concentrations and light near or at the edge of the absorption peak, it is possible to increase the depth effect and control it in a targeted way and in addition to utilize the bactericidal effect of the dye. By the choice of a wavelength outside or at the edge of the absorption peak of the dye employed, at concentrations of a suitable dye, enough energy is absorbed, even in deeper layers, to obtain activated oxygen, such as oxygen in the singlet state, in a clinically effective amount for an enhanced bactericidal effect. This effect can be enhanced still further by the choice of greater light intensity.

As a result, it is possible to put markedly more dye into interaction with the light and thus to enhance the bactericidal effect markedly.

The fact that this kind of change in the PDT method leads to attaining the object of the invention is surprising, especially since in general, the interaction between molecules and light, which is necessary here for oxygen activation in order to kill off the bacteria, also decreases drastically if the wavelength is shifted away from the absorption maximum.

According to claim 1, a system of dye and light unit should be selected in which the dye has an at least local absorption maximum at a first wavelength and the dye at least upon application is in a dye solution, and the maximum intensity of the light output by the light unit has a wavelength which is greater by at least 100 nm than the first wavelength, and that the dye solution has a dye concentration of greater than or equal to 0.1 weight %, and in particular of 1.0 weight %. By the choice of a wavelength spacing of at least 100 nm from the resonant wavelength of the dye at which the maximum absorption occurs, it is assured that an adequate depth effect can be achieved. The dye concentration of more than 0.1 weight %, conversely, assures an adequate energy absorption for activating the oxygen, even with incident light that is outside the absorption maximum. The light output can be determined easily by one skilled in the art with knowledge of the applied quantity of dye and of the absorption curves.

As a result of the higher dye concentration of the solution that is made possible by this method, rinsing of the treatment site provided with dye solution before the application of the light, which is otherwise usually necessary in order to attain the necessary low concentration, can be dispensed with. As a result, one work step is eliminated, and a defined, replicable dye distribution can be achieved.

The choice of a light source with maximum intensity at a higher wavelength than the absorption maximum has the advantage, over the choice of a light source with a lesser wavelength than the absorption maximum, that higher wavelengths are less harmful to the human or animal body, can usually be produced less expensively, and cause slighter thermal effects.

Numerous sources can be considered as the light source. The desired result can also be attained with a wide-band source. The energy efficiency, however, is very poor, since only certain parts of the spectrum contribute to the (depth) effect disclosed here. For instance, if light that also has energy at the absorption maximum of the dye employed is used, then this must be called a combination of what was previously known and the procedure according to the invention. The intensity that is output at the absorption maximum has almost solely a superficial effect (particularly at the higher concentrations selected here). The intensity that is located outside the absorption peak can penetrate more deeply and can have the effect intended by the invention. Intensity that is far from each absorption maximum will not develop any effect according to the invention but instead will contribute to the bactericidal effect solely by way of heating.

From the above it can be inferred that the radiation emitted by a wide-band source can be filtered without sacrificing effectiveness. This also prevents an overly strong illumination of the region to be treated. However, from this it can also be inferred that the choice of a light source with a small bandwidth or of monochromatic light is especially advantageous (claim 4). Examples that can be named here are light-emitting diodes, or lasers.

The term light is understood here to mean not solely light in the range visible to a human being. Instead, depending on the dye employed, light in the infrared or ultraviolet range can also be employed as light according to the invention.

According to claim 2, light can alternatively be selected that has its maximum intensity at a wavelength at which the absorption of the dye is reduced by at least 29%, and in particular more than 50%, compared to the absorption maximum.

By such a choice of the light, at which the maximum intensity is located outside the wavelength range at which the absorption has dropped by more than 1/√2 of the maximum absorption value, it is possible to assure adequate transmission (through the uppermost dye regions) even with very wide absorption peaks, or, with especially narrow-band absorbers, to assure an adequate interaction between light and dye. Depending on the depth effect or dye layer density desired, the choice of light outside FWHM (full width at half maximum), that is, outside the wavelength range at which the absorption amounts to 50% or more of the maximum absorption value, may also be especially advantageous.

Because of the location of the absorption maximum, the form of the absorption peak, and the compatibility with simultaneous bactericidal action at the concentrations used according to the invention, the use of methylene blue in accordance with claim 3 is especially advantageous.

Especially methylene blue is chosen as the dye, the use of light in accordance with claim 5 in the range of between 790 nm and 830 nm, preferably between 800 nm and 820 nm, and in particular of 810 nm, is especially advantageous, since then the absorption and transmission ratios allow treatment with an optimal depth effect. It is possible in this case to achieve an optimal energy distribution in typical thicknesses of tissue layers penetrated by dye, as well as optimal utilization of the incident energy. This is attained in treatment in the mouth particularly with a choice of a dye concentration of 1.0 weight %.

According to claim 6, the object is also attained by a device with a light unit, especially a laser unit, and a dye application unit, in which the light unit contains a light source with an emitted light wavelength in the range of from 790 nm to 830 nm, preferably from 800 nm to 820 nm, and in particular of 810 nm, and in which the dye application unit is equipped with dye, preferably a dye with a phenothiazine molecular structure, in particular methylene blue, as the dye to be applied.

A device of this kind is an implementation of a system in accordance with at least one of the foregoing claims and is especially well suited to employ the invention in practice.

The light unit may for instance contain a laser and in particular a semiconductor diode laser. However, any other kind of light source may be selected that meets the criteria of the claims with regard to its wavelength and if applicable its output. Advantageously, the light output can be selected variously, for instance beginning with an output of 0.1 W, followed by an intermediate stage of 1.0 W, and a maximum output of 2.5 or even 10 W.

In practice, a light output, in particular laser output, of between 0.1 W and 10 W in accordance with claim 7 is especially advantageous.

In addition, in the advantageous embodiment of a choice of methylene blue in aqueous solution in the concentration range recited in claim 8, a bactericidal effect intrinsic to the dye itself occurs, which—as studies by the inventor have shown—is apparent in this concentration even without further optical excitation by means of a laser.

In an advantageous feature, the dye application unit is formed as recited in claim 9. Accordingly, it may contain a storage tank, in particular a storage vial, and in addition a dispenser unit, operated with pressure, in particular hydraulically or pneumatically, for ejecting the dye from the storage tank onto the region to be treated. As the storage tank, a storage vial, of the kind that is commercially available and interchangeable, is especially preferable. Alternatively, the device may also include an vial specific to that equipment, which is refilled as needed and reinserted into the device.

Particularly in use in the mouth, it is especially advantageous, in accordance with claim 10, to use an atraumatic cannula for applying the dye.

Since it is entirely possible for the regions of the patient to be treated to be touched by this part of the device, an atraumatic cannula is especially advantageous for painless treatment or treatment with little pain.

In practice, it is also preferable either to use an optical waveguide with a core diameter of 100 μm, optionally 500 μm to 1000 μm, for working in tight spaces and crevices, or in accordance with claim 11, for irradiating larger areas, to use a collimated optical element equipped with an optical waveguide. Such an element preferably has a spot area of between 0.05 cm² and 4 cm², and possibly more, and even 5 cm² and more if the light output is especially high.

With such an optical waveguide, the light can be delivered to the region to be treated in a highly metered, targeted and precise way, and an unnecessary exposure to the light of regions other than those to be treated is avoided. Moreover, such an optical waveguide is easy for the treating physician or other person performing the treatment to manipulate.

The entire device is advantageously accommodated in a common housing. In particular, the housing can be portable, so that the entire device can easily be carried from one treatment location to another.

Finally, the dye application unit and the laser unit advantageously each have a handpiece by which a person performing the treatment can grasp it and move it about in a targeted way.

According to claim 12, the object is also attained by a method in which a dye solution is applied and irradiated with light; the dye in the dye solution has an at least local absorption maximum at a first wavelength, and the maximum intensity of the light has a wavelength which deviates by at least 100 nm from the first wavelength, in particular is greater by at least 100 nm than the first wavelength, and the dye solution has a concentration of the dye of greater than or equal to 0.1 weight %.

According to claim 13, light can alternatively be selected which has a maximum intensity at a wavelength at which the absorption of the dye is reduced by at least 29%, and in particular more than 50%, compared to the absorption maximum.

### BRIEF DESCRIPTION OF THE DRAWINGS

Further characteristics and advantages of the device according to the invention will become apparent from the ensuing description of one exemplary embodiment in conjunction with the accompanying drawings. Shown are:

FIG. 1, studies of absorption in methylene blue solutions;

FIG. 2, a schematic view of a device according to the invention in an exemplary embodiment.

### WAY(S) OF EMBODYING THE INVENTION

The drawings shown are purely schematic and in particular are not to scale. They serve the purpose solely of illustration and explanation and are not to be understood as the only construction drawing for a device according to the invention.

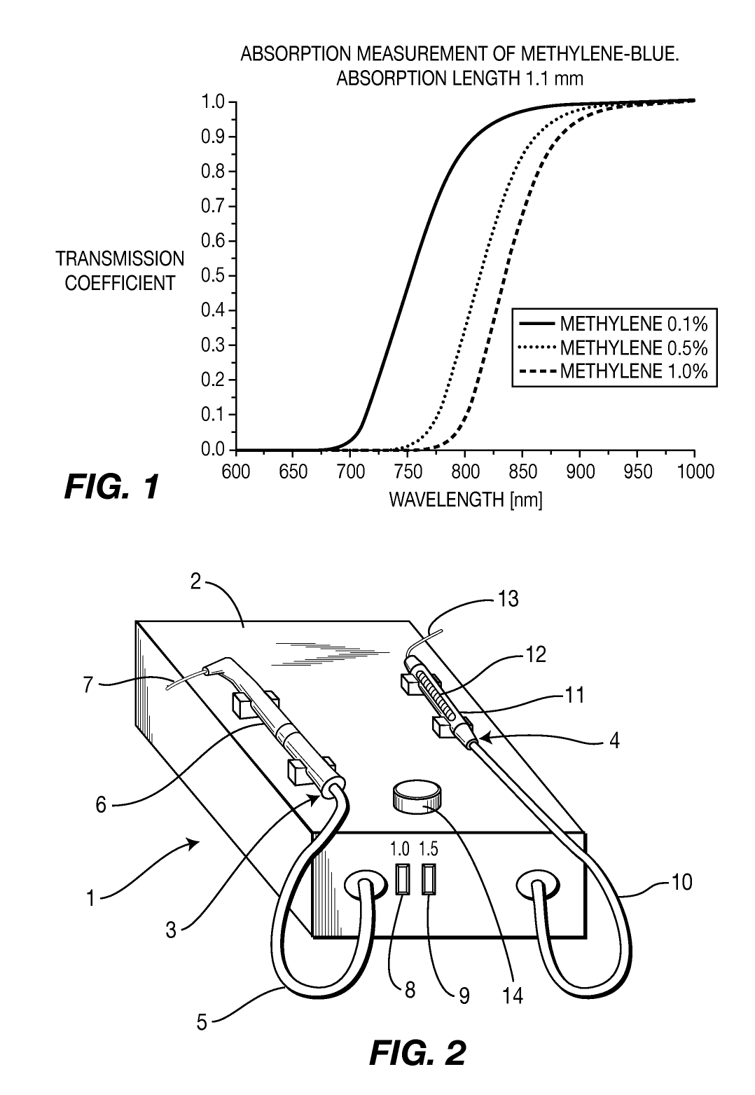

FIG. 1 shows studies of absorption at different concentrations of methylene blue solutions. From it, with knowledge of the absorption maximum of methylene blue, the effect of the invention can be understood. If light in the range of this maximum is selected, the transmission coefficient is quite low. As a result, the penetration depth of the light is quite limited as well, and hence an interaction between light and dye that enhances the bactericidal effect is possible only the vicinity of the surface.

In FIG. 2, a treatment device is identified overall by reference numeral 1. It has a housing 2, in or on which a laser unit 3 and a dye application unit 4 are jointly disposed. The housing can be connected to a conventional electrical voltage supply by way of an energy supply, not shown here, and in its interior, it contains devices for distributing the electrical energy to the various components. The semiconductor laser diode with triggering and cooling is also accommodated in the housing 2.

The two essential components, the laser unit and the dye application unit 4, will now be described in further detail. This semiconductor laser diode, in this exemplary embodiment, is a diode that emits laser light at a wavelength of 810 nanometers. First, it must be seen that the base 3 of the handpiece is connected to the semiconductor laser diode via a flexible optical waveguide. Alternatively, the diode may be accommodated in the handpiece 6 itself and connected electrically to the housing for supply purposes. The light of the semiconductor laser diode is fed in the interior of the handpiece 5 into a further optical waveguide, one end of which protrudes in the form of a treatment tip 7 from the handpiece 6 and by way of which the laser light can be steered in a targeted way to the region to be treated. In the exemplary embodiment shown, this optical waveguide is an optical fiber that is disposed replaceably in the device. As possible optical fibers, fibers with diameters of 100 micrometers to 1000 micrometers, preferably fibers from 200 micrometers to 600 micrometers, are used here. Alternatively, collimated optical elements with irradiation areas of from 0.5 cm² to 5 cm², with various geometries, can be mounted on the handpiece.

A switch not identified by reference numeral is disposed on the handpiece 6 here for switching the laser on and off during the treatment. As another possibility for switching the laser on and off, a foot switch connected to the housing may be employed. Two pushbuttons 8 and 9 are also disposed on the housing; with them, the laser output can be preselected, in this case as 1.0 W or 1.5 W.

The dye application unit 4 likewise includes a flexible connection cable 10, by way of which a handpiece 11 is connected to the housing 2. A dye vial 12 is disposed in the handpiece 6 and contains methylene blue at a concentration of 0.1 to 1.0 weight %, dissolved in water. On a treatment end, the handpiece 11 discharges into an atraumatic cannula 13, by means of which the dye solution containing methylene blue can be applied to the area of the patient that is to be treated.

Another part of the dye application unit 4, not shown here because it is located inside the housing, is a pump system, which is capable of generating air pressure or hydraulic pressure to be applied to the inside of the dye vial 12 through the flexible connection cable 10. Finally, a switch or button not identified by reference numeral is disposed on a handpiece 11, and by way of which the operation of the dye application unit can be initiated or stopped. The electrical signals of this switch are likewise carried via the flexible cable 10 into the housing and from there to the affected devices.

Finally, the treatment device 1 also includes an emergency off switch 14, with which if needed the operation of the entire device can be stopped.

Although in principle all the regions of a patient can be treated bactericidally, but extracorporeal treatment is also possible, with the device of the invention, this device is especially directed to the treatment in the mouth of a patient, especially for treating the gums.

This device is operated as follows:

A physician or other person performing the treatment, by means of the dye application unit 4, first applies the dye solution to the region to be treated, especially a tooth region or gum region. To that end, he grasps the dye application unit 4 by its handpiece 11 and guides the atraumatic cannula 13 as far as the region to be treated. Actuating the switch or button disposed on the handpiece 11 trips the expulsion of dye through the atraumatic cannula 13. With his free hand, the treating physician or other person performing the treatment can now grasp the handpiece 6 of the laser unit 3, having beforehand, using the pushbutton 8 or 9, selected the desired laser output of 1.0 W or 1.5 W. The treating physician guides the handpiece 6 in such a way that the treatment tip 7 of the optical waveguide points to the region to be treated, which has already been supplied with the dye solution. The person performing the treatment can also operate the appropriate switch or button on the handpiece 6 and begin the laser treatment for the bactericidal action.

In this treatment, it has been proved to be useful, after the conclusion of the laser treatment, not to rinse out the dye or dye solution still located in the vicinity of the regions to be treated, but instead to leave it there, so that over time, until the dye is removed naturally, a further bactericidal effect can be attained.

### LIST OF REFERENCE NUMERALS

1 Treatment device
2 Housing
3 Laser unit
4 Dye application unit
5 Flexible optical waveguide
6 Handpiece
7 Treatment tip
8 Pushbutton
9 Pushbutton
10 Connection cable
11 Handpiece
12 Dye vial
13 Atraumatic cannula
14 Emergency off switch

1. A system for the antibacterial treatment of a human or animal body, comprising:
a dye, in which the dye has an at least local absorption maximum and a first wavelength and at least upon application the dye is in the form of a dye solution, and
a light unit, for the generation and directional outputting of light onto the regions to be treated,
wherein a maximum intensity of the light output by the light unit has a wavelength which is greater by at least 100 nm than the first wavelength; and that the dye solution has a dye concentration of greater than or equal to 0.1 weight.

2. A system for the antibacterial treatment of a human or animal body comprising:
a dye, in which the dye has an at least local absorption maximum and a first wavelength and at least upon application the dye is in the form of a dye solution, and
a light unit for the generation and for directionally outputting light onto the regions to be treated,
wherein a maximum intensity of the light output by the light unit has a wavelength, greater than the first wavelength, at which the absorption of the dye compared to the absorption maximum is reduced by at least 29% and that the dye solution has a dye concentration of greater than or equal to 0.1 weight %.

3. The system as defined by claim 1 wherein the dye is methylene blue.

4. The system as defined by claim 1 wherein the light unit for generating the light includes a monochromatic light source.

5. The system as defined by claim 3, wherein the light unit has a light source for generating light at a wavelength of between 790 nm and 830 nm.

6. A device for the antibacterial treatment of a patient comprising:
a light unit for the generation and directional outputting of laser light onto regions to be treated, and
a dye application unit for the application of dye, to the regions to be treated,
wherein the light unit includes a light source, which emits light at a wavelength in the range of from 790 nm to 830 nm, and that the dye application unit is equipped with dye, preferably with dye in water with a concentration of 0.1 weight % or more.

7. The device as defined by claim 6, wherein the light unit includes a laser with a laser power of from 0.1 W to 10 W.

8. The device as defined by claim 6, wherein the dye is contained in the dye application unit in a concentration in aqueous solution of between 0.75 weight % and 1.25 weight %.

9. The device as defined by claim 6 wherein the dye application unit contains a storage tank, with the dissolved dye as well as a dispenser unit, operated with pressure for ejecting the dye from the storage tank onto a region to be treated.

10. The device as defined by claim 6 wherein the dye application unit has a dispenser end which has an atraumatic cannula.

11. The device as defined by claim 6 wherein the light unit has a collimated optical element with irradiation areas of 0.05 cm² to 5 cm², or an optical waveguide with a diameter of from 100 μm to 1000 μm for conducting the light into the region to be treated.

12. A method for the antibacterial treatment of a human or animal body, in which a dye solution is applied and irradiated with light, and the dye in the dye solution has an at least local absorption maximum at a first wavelength, wherein the maximum intensity of the light has a wavelength which deviates by at least 100 nm from the first wavelength, and that the dye solution has a dye concentration of greater than or equal to 0.1 weight %.

13. A method for the antibacterial treatment of a human or animal body in which a dye solution is applied and irradiated with light, and the dye in the dye solution has an at least local absorption maximum at a first wavelength, wherein a maximum intensity of the light has a wavelength at which the absorption of the dye compared to the absorption maximum is reduced by at least 29% and that the dye solution has a dye concentration of greater than or equal to 0.1 weight %.

14. The system according to claim 1 in which the dye has a phenothiazine molecular structure.

15. The system according to claim 1 wherein the dye solution has a dye concentration of 1.0 weight %.

16. The system according to claim 2 wherein the absorption of the dye compared to the absorption maximum is reduced by more than 50%; and that the dye solution has a dye concentration of 1.0 weight %.

17. The system as defined by claim 3, wherein the light unit has a light source for generating light at a wavelength of between 800 nm and 820 nm.

18. The system as defined by claim 3, wherein the light unit has a light source for generating light at a wavelength of 810 nm.

19. The device according to claim 6 wherein the light unit comprises a laser unit.

20. The device according to claim 6 wherein the dye application unit is for the application of a dye in solution form.
